(19) United States
(12) Patent Application Publication
(10) Pub. No.: US 2014/0357595 A1
(43) Pub. Date: Dec. 4, 2014

(54) METHODS OF PREVENTING AND TREATING RECURRENCE OF A HEPATITIS C VIRUS INFECTION IN A SUBJECT AFTER THE SUBJECT HAS RECEIVED A LIVER TRANSPLANT

(71) Applicant: Gilead Pharmasset LLC, Foster City, CA (US)

(72) Inventors: Jill M. Denning, Raleigh, NC (US); John George McHutchison, Woodside, CA (US); G. Mani Subramanian, Los Altos Hills, CA (US); William Thomas Symonds, III, San Francisco, CA (US)

(73) Assignee: Gilead Pharmasset LLC, Foster City, CA (US)

(21) Appl. No.: 14/293,876

(22) Filed: Jun. 2, 2014

Related U.S. Application Data
(60) Provisional application No. 61/831,107, filed on Jun. 4, 2013.

(51) Int. Cl. A61K 31/7072; A61K 45/06; A61K 31/4184; A61K 31/7056

(52) U.S. Cl. CPC A61K 31/7072; A61K 31/7056; A61K 45/06; A61K 31/4184
USPC 514/51

(57) ABSTRACT

This application describes methods of preventing, treating or reducing the risk of recurrence of a hepatitis C virus infection in a subject after the subject has received a liver transplant. Disclosed herein are methods of preventing or reducing the risk of recurrence of a hepatitis C virus infection in a subject after the subject has received a liver transplant, the methods comprising administering to the subject an effective amount of Compound 1.

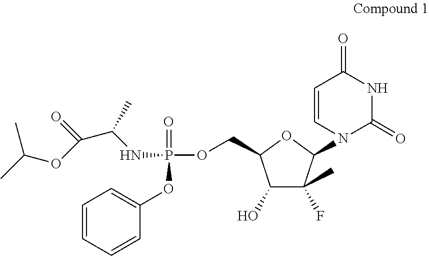 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

Also disclosed are methods of reducing HCV RNA levels to about 25 IU/mL or lower in a subject having received a liver transplant comprising administering to the subject an effective amount of Compound 1.

This application claims the benefits of U.S. Provisional Application 61/831,107, filed on Jun. 4, 2013, which is incorporated herein by reference in its entirety.

### FIELD

This application relates to methods of preventing, treating or reducing the risk of recurrence of a hepatitis C virus infection in a subject after the subject has received a liver transplant.

### BACKGROUND

An estimated 170-180 million people are chronically infected with hepatitis C virus (HCV) worldwide (Ghany et al., “Diagnosis, Management, and Treatment of Hepatitis C: An Update,” Hepatology (2009) 49(4):1335-74). In the United States (US), an estimated 3 million people have chronic HCV infection (Dienstag et al., “American Gastroenterological Association Technical Review on the Management of Hepatitis C,” Gastroenterology (2006) 130(1):231-64). Chronic HCV infection can cause chronic liver disease, cirrhosis, liver failure, hepatocellular carcinoma (HCC) and death. The complications of chronic HCV account for the most common indication for liver transplantation in the US.

After liver transplantation, recurrence of HCV infection occurs in almost all subjects (Feray C, Samuel D, Thiers V, Gigou M, Pichon F, Bismuth A, et al. Reinfection of liver graft by hepatitis C virus after liver transplantation. J. Clin. Invest. (1992) 89 (4):1361-5) and is associated with reduced graft and patient survival (Prieto M, Berenguer M, Rayon J M, Cordoba J, Arguello L, Carrasco D, et al. High incidence of allograft cirrhosis in hepatitis C virus genotype 1b infection following transplantation: relationship with rejection episodes. Hepatology (1999) 29 (1):250-6; Berenguer M. Natural history of recurrent hepatitis C. Liver Transpl. (2002) 8 (10 Supp. 1):S14-8). Recurrence of infection is immediate in recipients who are serum HCV RNA-positive at liver transplantation (LT).

Recurrent liver disease is more aggressive after LT than in immunocompetent subjects, as progression to cirrhosis at five years occurs in 10-50% of LT subjects (Picciotto F P, Tritto G, Lanza A G, Addario L, De Luca M, Di Costanzo G G, et al. Sustained virological response to antiviral therapy reduces mortality in HCV reinfection after liver transplantation. J. Hepatol. (2007) 46 (3):459-65; Berenguer M, Liver Transpl. (2002)). Once cirrhosis is established, the probability of liver graft failure is 42% within 12 months (Berenguer M, Prieto M, San Juan F, Rayon J M, Martinez F, Carrasco D, et al. Contribution of donor age to the recent decrease in patient survival among HCV-infected liver transplant recipients. Hepatology (2002) 36 (1):202-10).

Anti-viral therapies based on pegylated interferon-alpha (PEG) and ribavirin (RBV) may be indicated after liver transplantation (pre-emptive therapy) or later once chronic hepatitis has been confirmed and immunosuppression is relatively low. This therapy is limited, however, by poor tolerance, poor efficacy and severe adverse reactions.

The development of an IFN-free regimen for the treatment of chronic HCV infection has the potential to make a major impact on the global incidence, prevalence and burden of disease due to HCV infections. New treatment options are especially crucial in patient populations for whom treatment with PEG-IFN is not possible, is undesired, or lacks sufficient efficacy such as end-stage liver disease and liver transplantation.

### SUMMARY

Disclosed herein are methods of preventing or reducing the risk of recurrence of a hepatitis C virus infection in a subject after the subject has received a liver transplant, the methods comprising administering to the subject an effective amount of Compound 1. Also disclosed are methods of preventing or reducing the risk of recurrence of a hepatitis C virus infection in a subject after the subject has received a liver transplant, the methods comprising providing to the subject a 5′-mono-, di- or triphosphate metabolite of Compound 1. Also disclosed are methods of reducing HCV RNA levels to lower than about 25 IU/mL in a subject having received a liver transplant comprising administering to the subject an effective amount of Compound 1. Also disclosed are methods of reducing HCV RNA levels to lower than about 25 IU/mL in a subject having received a liver transplant comprising providing to the subject a 5′-mono-, di- or triphosphate metabolite of Compound 1. Also disclosed are methods of treating a recurrent hepatitis C virus infection in a subject after the subject has received a liver transplant, comprising administering to a subject in need thereof an effective amount of Compound 1. Also disclosed are methods of treating a recurrent hepatitis C virus infection in a subject after the subject has received a liver transplant, comprising providing to a subject infected with hepatitis C virus the 5′-mono-, di- or triphosphate metabolite of Compound 1.

Also provided is the use of Compound 1 in the manufacture of a medicament for preventing or reducing the risk of recurrence of a hepatitis C virus infection in a subject after the subject has received a liver transplant. Also provided is the use of Compound 1 in the manufacture of a medicament for reducing HCV RNA levels to lower than about 25 IU/mL in a subject having received a liver transplant. Also provided is the use of Compound 1 in therapy for preventing or reducing the risk of recurrence of a hepatitis C virus infection in a subject after the subject has received a liver transplant. Also provided is the use of Compound 1 in therapy for reducing HCV RNA levels to lower than about 25 IU/mL in a subject having received a liver transplant.

In certain methods, Compound 1 is administered for a pre-transplant duration before the subject has received a liver transplant. This pre-transplant duration can be from about 1 week to about 48 weeks. In other methods, Compound 1 is administered for a post-transplant duration after the subject has received a liver transplant. The post-transplant duration can be from about 1 week to about 48 weeks.

Certain methods further comprise administering to the subject an effective amount of one or more additional anti-HCV agents. In certain methods, the additional antiviral agent can be ribavirin. In certain methods, the additional antiviral agent can be an HCV NS5A protein inhibitor, a non-nucleotide NS5B polymerase inhibitor, or an NS3 inhibitor. Additional aspects of the disclosed methods are described below.

### DETAILED DESCRIPTION

The abbreviation “ALT” refers to alanine aminotransferase. Increased serum ALT levels can accompany hepatocellular injury or necrosis of striated muscle. In addition, release of ALT from the cytosol may occur secondary to cellular necrosis or as a result of cellular injury with membrane damage. An increased serum ALT level is one indication of hepatic damage.

The abbreviation “AST” refers to aspartate aminotransferase, an enzyme normally found in the blood in low levels. Increased serum AST may result from disease or damage to an organ, including the liver. An increased serum AST level is one indication of hepatic damage.

The term “body mass index” (BMI) refers to the ratio of a subject's weight to the subject's height. BMI is expressed in units of kg/m² and is calculated as follows:

$$BMI = \frac{weight ( pounds ) \times 703}{{( height in inches )}^{2}} or \frac{weight in kilograms}{{( height in meters )}^{2}}$$

The Child-Pugh (CPT) score, which may also be referred to as the Child-Pugh Turcotte score, is measured on a scale of 1-15 and is used to assess the prognosis of chronic liver disease in subjects with cirrhosis.

The terms “infected with HCV,” “HCV infection” and similar terms refer to a documented HCV infection, including acute and chronic infection. HCV infection can be shown, for instance, by a positive anti-HCV antibody test, HCV RNA, or HCV genotyping test.

HCV Viral Load (HCV RNA test, Quantitative) refers to the detection and measurement of the number of viral RNA particles in blood.

Viral genotyping is used to determine the kind, or genotype, of the HCV virus present. There are at least 6 major types of HCV (GT 1-6); the most common in the United States is genotype 1. The abbreviation “GT” refers to genotype.

The abbreviation “IL28B” refers to interleukin 28B, which is one isoform of the interleukin-28 (IL28) cytokine. A single nucleotide polymorphism (SNP) in the IL28B promoter region was identified and is used to predict response to interferon treatment in subjects with HCV.

The abbreviation “IU” refers to international unit, which is a measure of the amount of a substance based on its biological activity or effect.

The abbreviation “LLOQ” refers to the lower limit of quantification. As used herein in reference to HCV RNA measurements, the LLOQ is about 25 IU/mL (as measured by a Roche Cobas® Taqman® Version V2.0 Assay for HCV).

The Milan criteria are applied as a basis for selecting subjects with cirrhosis and hepatocellular carcinoma for liver transplantation. The Milan criteria are defined as follows: (1) a single lesion with a maximum size of 5 cm or (2) as many as 3 lesions with the largest not exceeding 3 cm; and (3) no vascular invasion (Mazzaferro et al., “Liver Transplantation for the Treatment of Small Hepatocellular Carcinomas in Patients with Cirrhosis,” N. Engl. J. Med. (1996) 334:693-700).

The Model for End-Stage Liver Disease (MELD) score, measured on a scale of 6 to 40, is used for adult liver transplant candidates and is calculated based on bilirubin, international normalized ratio (INR) and creatinine laboratory test results.

The terms “pre-transplant duration” and “post-transplant duration” refer, respectively, to durations of therapy that occur before the subject is to receive a liver transplant, and after the subject has received a liver transplant.

The terms “prevent” and “preventing” refer to causing the clinical symptoms of the disease not to develop.

The term “subject” refers to a human.

The abbreviation “RVR” refers to rapid virologic response (see, for example, Poordad et al., Clin. Infect. Dis. (2008) 46:78-84).

A sustained virologic response (SVR) for a subject treated according to one of the methods described herein refers to the subject having HCV RNA less than the lower limit of quantification (LLOQ, for example, about 25 IU/mL) for or at a period of time post-treatment, as measured in accordance with the assay methodology described herein.

The abbreviation q.d. means that a dose is administered once a day.

The abbreviation b.i.d. means that the dose is administered twice a day. The term “divided b.i.d.,” as used herein, means that a total dose amount is divided into two doses administered at different times during the course of a day. The two “divided b.i.d.” doses may be equal or different.

Compound 1, (S)-isopropyl 2-(((S)-(((2R,3R,4R,5R)-5-(2,4-dioxo-3,4-dihydropyrimidin-1(2H)-yl)-4-fluoro-3-hydroxy-4-methyltetrahydrofuran-2-yl)methoxy)(phenoxy)phosphoryl)amino)propanoate, is represented by the following chemical structure:

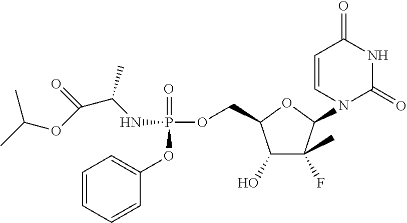 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

(see, for example, U.S. Pat. No. 7,964,580).

Compound 1 is converted in vivo to its 5′-mono-, di- and triphosphate metabolites (Id.), represented by the following chemical structures:

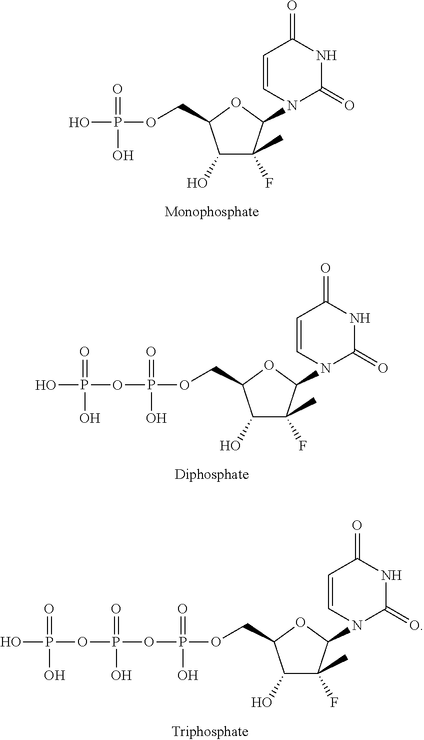 [SMILES: C[C@@]1(F)[C@H](O)[C@@H](COP(=O)(O)O)O[C@H]1n1ccc(=O)[nH]c1=O ; C[C@@]1(F)[C@H](O)[C@@H](COP(=O)(O)OP(=O)(O)O)O[C@H]1n1ccc(=O)[nH]c1=O ; C[C@@]1(F)[C@H](O)[C@@H](COP(=O)(O)OP(=O)(O)OP(=O)(O)O)O[C@H]1n1ccc(=O)[nH]c1=O]

The 5′-triphosphate metabolite of Compound 1 is a potent and selective inhibitor of HCV replication mediated by the HCV NS5B RNA dependent RNA polymerase (Sofia et al., J. Med. Chem. (2010) 53(19):7202-7218).

Compound 1 has demonstrated a low incidence of resistance and has been well-tolerated in clinical trials. Compound 1, also known as “sofosbuvir,” is currently marketed under the trademark Sovaldi®.

Various methods further comprise administering to the subject one or more additional anti-HCV agents. Such additional anti-HCV agents include ribavirin, ribavirin analogs, interferons, NS3 protease inhibitors, NS5a inhibitors, NS5b polymerase inhibitors, alpha-glucosidase 1 inhibitors, cyclophilin inhibitors, hepatoprotectants, other nucleoside inhibitors of HCV, non-nucleoside inhibitors of HCV, other drugs for treating HCV. Suitable additional anti-HCV agents can be selected based on the condition to be treated, cross-reactivities of ingredients and pharmaco-properties of the combination.

Examples of additional anti-HCV agents include, without limitation, the following:
A. Ribavirin and ribavirin analogs, for example, Copegus®, Rebetol®, Ribasphere®, Vilona®, and Virazole®, VX-497, and viramidine (taribavirin);
B. Interferons, for example, pegylated rIFN-alpha 2b (PEG-Intron), pegylated rIFN-alpha 2a (Pegasys), rIFN-alpha 2b (Intron A), rIFN-alpha 2a (Roferon-A), interferon alpha (MOR-22, OPC-18, Alfaferone, Alfanative, Multiferon, subalin), interferon alfacon-1 (Infergen), interferon alpha-nl (Wellferon), interferon alpha-n3 (Alferon), interferon-beta (Avonex, DL-8234), interferon-omega (omega DUROS, Biomed 510), albinterferon alpha-2b (Albuferon), IFN alpha XL, BLX-883 (Locteron), DA-3021, glycosylated interferon alpha-2b (AVI-005), PEG-Infergen, PEGylated interferon lambda (PEGylated IL-29), or belerofon, IFN alpha-2b XL, rIFN-alpha 2a, consensus IFN alpha, infergen, rebif, pegylated IFN-beta, oral interferon alpha, feron, reaferon, intermax alpha, r-IFN-beta, and infergen+actimmuneribavirin and
C. NS5A inhibitors, for example, Compound A (described below), Compound B (described below), GS-5800, ABT-267, JNJ-47910382, daclatasvir (BMS-790052), ABT-267, MK-8742, EDP-239, IDX-719, PPI-668, GSK-2336805, ACH-3102, A-831, A-689, AZD-2836 (A-831), AZD-7295 (A-689), and BMS-790052;
D. NS5B polymerase inhibitors, for example, GS-9669, ABT-333, ABT-072, tegobuvir (GS-9190), TMC647055, setrobuvir (ANA-598), filibuvir (PF-868554), VX-222, IDX-375, IDX-184, IDX-102, BI-207127, valopicitabine (NM-283), PSI-6130 (R1656), PSI-7851, BCX-4678, nesbuvir (HCV-796), BILB 1941, MK-0608, NM-107, R7128, VCH-759, GSK625433, XTL-2125, VCH-916, JTK-652, MK-3281, VBY-708, A848837, GL59728, A-63890, A-48773, A-48547, BC-2329, BMS-791325, and BILB-1941;
E. NS3 protease inhibitors, for example, GS-9857, GS-9451, GS-9256, ABT-450, simeprevir (TMC-435), boceprevir (SCH-503034), narlaprevir (SCH-900518), vaniprevir (MK-7009), MK-5172, danoprevir (ITMN-191), sovaprevir (ACH-1625), neceprevir (ACH-2684), Telaprevir (VX-950), VX-813, VX-500, faldaprevir (BI-201335), asunaprevir (BMS-650032), BMS-605339, VBY-376, PHX-1766, YH5531, BILN-2065, and BILN-2061;
F. alpha-glucosidase 1 inhibitors, for example, celgosivir (MX-3253), Miglitol, and UT-231B;
G. hepatoprotectants, for example, IDN-6556, ME 3738, MitoQ, and LB-84451; H. non-nucleoside inhibitors of HCV, for example, benzimidazole derivatives, benzo-1,2,4-thiadiazine derivatives, and phenylalanine derivatives; and
I. other anti-HCV agents, for example, zadaxin, nitazoxanide (alinea), BIVN-401 (virostat), DEBIO-025, VGX-410C, EMZ-702, AVI 4065, bavituximab, oglufanide, PVN-17, KPE02003002, actilon (CPG-10101), KRN-7000, civacir, GI-5005, ANA-975, XTL-6865, ANA 971, NOV-205, tarvacin, EHC-18, and NIM811.

Ribavirin is a guanosine analogue marketed under the trademark COPEGUS® and described in the Merck Index (12th Edition), monograph no. 8365. Ribavirin, 1-β-D-ribofuranosyl-1H-1,2,4-triazole-3-carboxamide, is represented by the following chemical structure:

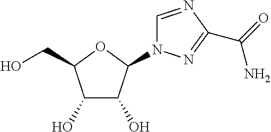 [SMILES: NC(=O)c1ncn([C@@H]2O[C@H](CO)[C@@H](O)[C@H]2O)n1]

(see, for example, U.S. Pat. No. 4,530,901).

Compound A is an inhibitor of the HCV NS5A protein and is represented by the following chemical structure:

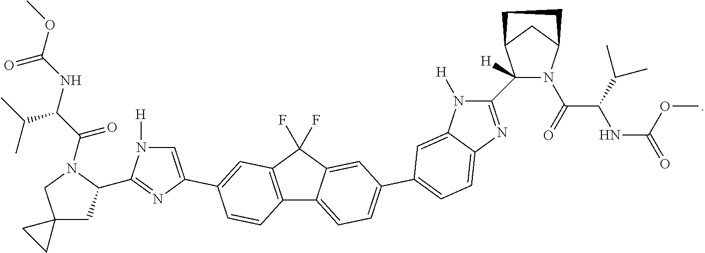

(see, for example, U.S. Patent Application Pub. No. 20100310512 A1).

Compound B is an NS5A inhibitor and is represented by the following chemical structure:

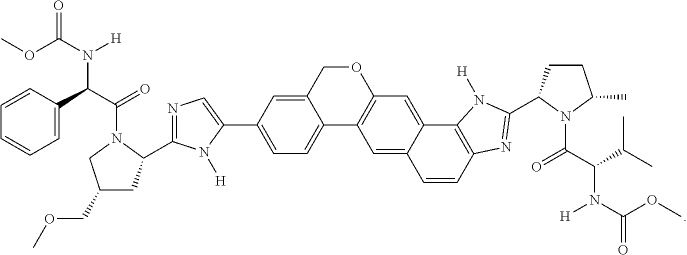

In some methods, Compound 1 and Compound A are administered to the subject. In other methods, Compound 1 and Compound B are administered to the subject. In some of these methods, ribavirin is also administered to the subject. For example, in some methods Compound 1, Compound A and ribavirin are administered to the subject.

This application also describes various methods of preventing, treating and reducing the risk of recurrence of a hepatitis C virus infection in a subject after the subject has received a liver transplant.

One method provides for preventing or reducing the risk of recurrence of a hepatitis C virus infection in a subject after the subject has received a liver transplant, comprising administering to the subject an effective amount of Compound 1. Another method provides for preventing or reducing the risk of recurrence of a hepatitis C virus infection in a subject after the subject has received a liver transplant, comprising providing to the subject a 5′-mono-, di- or triphosphate metabolite of Compound 1.

Another method provides for reducing HCV RNA levels to lower than about 25 IU/mL in a subject having received a liver transplant comprising administering to the subject an effective amount of Compound 1. Another method provides for reducing HCV RNA levels to lower than about 25 IU/mL in a subject having received a liver transplant comprising providing to the subject a 5′-mono-, di- or triphosphate metabolite of Compound 1.

Another method provides for maintaining HCV RNA levels to lower than about 25 IU/mL in a subject having received a liver transplant comprising administering to the subject an effective amount of Compound 1. Another method provides for maintaining HCV RNA levels to lower than about 25 IU/mL in a subject having received a liver transplant comprising providing to the subject a 5′-mono-, di- or triphosphate metabolite of Compound 1. HCV RNA levels relating to such reducing or maintaining may be measured at or for a given time period after the duration of therapy (for example, the duration of administration of Compound 1, that is, after the last day on which Compound 1 was administered to the subject). For example, in methods of reducing HCV RNA levels to lower than about 25 IU/mL, HCV RNA levels can be measured at a time period after the duration of administration of Compound 1. Such time period may be from about 1 week to about 48 weeks, for example about 2 weeks, about 4 weeks, about 8 weeks, about 10 weeks, about 12 weeks, or about 24 weeks after the duration of administration of Compound 1. For example, methods provide for reducing an HCV RNA level to lower than about 25 IU/mL or lower in a subject 2 to 24 weeks after the duration of administration of Compound 1. By further example, one method provides for reducing an HCV RNA level to lower than about 25 IU/mL or lower in a subject 12 weeks after the duration of administration of Compound 1. Yet other methods provide for treating hepatitis C virus infection, reducing HCV RNA levels to lower than about 25 IU/mL or lower, or maintaining HCV RNA levels below about 25 IU/mL in a post-transplant subject comprising providing to the subject a 5′-mono-, di- or triphosphate metabolite of Compound 1. Such reducing or maintaining may be at or for a time period after the duration of therapy (after the duration of providing the metabolite to the subject) as described above.

Another method provides for treating a recurrence of a hepatitis C virus infection in a subject after the subject has received a liver transplant, comprising administering to a subject in need thereof an effective amount of Compound 1. Another method provides for treating a post-liver transplant recurrent of hepatitis C virus infection, comprising providing to a subject infected with hepatitis C virus the 5′-mono-, di- or triphosphate metabolite of Compound 1.

In some methods, Compound 1 is administered to the subject for a pre-transplant duration (that is, administered before the subject has received or is to receive a liver transplant). In other methods, Compound 1 is administered for a post-transplant duration 356375.1 (that is, after the subject has received a liver transplant).

In the various methods, the pre-transplant duration or the post-transplant duration can be from about 1 week to about 48 weeks, which includes any and all intervening durations. For example, the pre-transplant duration or the post-transplant duration can be about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, or about 48 weeks. In some aspects, the pre-transplant duration or the post-transplant duration is 4 weeks. In some aspects, the pre-transplant duration or the post-transplant duration is 8 weeks. In some aspects, the pre-transplant duration or the post-transplant duration is 12 weeks. In other aspects, the duration is 24 weeks. In some aspects, the duration is 48 weeks.

The effective amount of Compound 1 can be from about 100 mg to about 800 mg per day. For example, the effective amount of Compound 1 can about 200 mg, about 300 mg, about 400 mg, about 500 mg, about 600 mg, about 700 mg, or about 800 mg per day. In some aspects, the effective amount of Compound 1 is 400 mg per day.

In various aspects, the methods comprise administering Compound 1 to the subject q.d. or b.i.d. For example, certain methods comprise administering to the subject about 400 mg per day of Compound 1 q.d.

In certain aspects, an effective amount of ribavirin can optionally be administered to the subject. In certain aspects, no ribavirin is administered. In other aspects, the effective amount of ribavirin is from about 100 mg to about 1400 mg per day. In certain aspects, the effective amount of ribavirin is from about 1000 mg to about 1200 mg per day. In certain aspects, the effective amount of ribavirin is based on the subject's body weight. For example, certain methods comprise administering to the subject either about 1000 mg or about 1200 mg per day of ribavirin based on the subject's body weight. Certain aspects comprise increasing the daily amount of ribavirin during the course of the duration. For example, certain methods comprise administering to the subject from about 100 mg to about 400 mg per day of ribavirin at the start of the duration, followed by increasing the daily amount of ribavirin up to a maximum amount of from about 200 mg to about 1400 mg per day. Certain aspects comprise administering to the subject about 400 mg per day of ribavirin at the start of the duration, followed by increasing the daily amount of ribavirin up to a maximum amount of from about 1000 mg to 1200 mg per day. In certain aspects, the daily amount of ribavirin is increased by 200 mg/day/month up to 1000 mg/day or 1200 mg/day, based on the subject's body weight. For example, certain methods comprise administering to the subject about 400 mg per day of ribavirin, followed by increasing the amount of ribavirin by 200 mg/day/month up to a maximum amount of 1000 mg or 1200 mg per day, based on the subject's body weight. Other aspects comprise decreasing the daily amount of ribavirin during the course of the duration. For example, certain methods comprise administering to the subject up to about 1200 mg/day at the start of the duration and decreasing the daily amount of ribavirin by 200 mg/day. Other methods comprise administering to the subject about 1400 mg/day at the start of the duration and decreasing the daily amount of ribavirin by 400 mg/day.

In some aspects, ribavirin is administered to the subject q.d. or b.i.d. For example, in certain methods ribavirin is administered to the subject b.i.d. As a further example, certain aspects comprise administering to the subject from about 1000 mg to about 1200 mg per day of ribavirin, divided b.i.d. For example, certain methods comprise administering to the subject about 1000 mg or about 1200 mg per day of ribavirin, based on the subject's body weight, divided b.i.d. Methods may comprise providing to the subject the 5′-mono-, di- and/or triphosphate metabolite of Compound 1 and RBV by administering to the subject Compound 1 and ribavirin according to the foregoing dosage amounts and frequencies.

Some aspects comprise administering to the subject from about 100 mg to about 800 mg per day of Compound 1 and from about 100 mg to about 1400 mg per day of ribavirin. For example, in certain aspects the methods comprise administering to the subject about 400 mg per day of Compound 1 and about 400 mg per day, about 600 mg per day, about 800 mg per day, about 1000 mg per day, or about 1200 mg per day of ribavirin. In certain aspects, the methods comprise administering to the subject about 400 mg per day of Compound 1 and about 1000 mg to about 1200 mg per day of ribavirin. In other aspects, the methods comprise administering to the subject about 400 mg per day of Compound 1, and about 1000 mg or about 1200 mg per day of ribavirin, wherein the amount of ribavirin is based on the subject's body weight. In additional aspects, the methods comprise administering to the subject about 400 mg per day of Compound 1 and an initial amount of about 400 mg per day of ribavirin, followed by administering to the subject about 400 mg per day of Compound 1 and increasing the ribavirin amount by 200 mg/day/month up to a maximum amount of about 1000 mg or about 1200 mg per day, depending on the subject's body weight. In further aspects, the methods comprise administering the effective amount of Compound 1 q.d. and the effective amount of ribavirin divided into multiple amounts (for example, b.i.d.). For example, in certain aspects, the methods comprise administering to the subject about 400 mg per day of Compound 1 q.d. and about 1000 mg or about 1200 mg per day of ribavirin, based on the subject's body weight, divided b.i.d. In other aspects, the methods comprise administering to the subject about 400 mg per day of Compound 1 q.d. and an initial amount of about 400 mg per day of ribavirin divided b.i.d., followed by administering to the subject about 400 mg per day of Compound 1 q.d. and increasing the ribavirin amount by 200 mg/day/month up to a maximum amount of about 1000 mg or about 1200 mg per day, depending on the subject's body weight, divided b.i.d. Methods may comprise providing to the subject the 5′-mono-, di- and/or triphosphate metabolite of Compound 1 and RBV by administering to the subject Compound 1 and ribavirin according to the foregoing dosage amounts and frequencies.

As described above, certain aspects provide for maintaining the HCV RNA levels in a subject below about 25 IU/mL. In certain methods wherein Compound 1 is administered before the subject has received a liver transplant, the subject has HCV RNA levels below about 25 IU/mL at about 1 week post-treatment (which is also about 1 week after having received the liver transplant). In other such methods, the subject has HCV RNA levels below about 25 IU/mL at about 2 weeks, about 4 weeks, about 8 weeks, about 12 weeks, about 24 weeks, or about 48 weeks post-treatment. In certain methods wherein Compound 1 is administered after the subject has received a liver transplant, the subject has HCV RNA levels below about 25 IU/mL at about 1 week post-treatment (i.e. about 1 week after the last dose of Compound 1 is administered). In other such methods, the subject has HCV RNA levels below about 25 IU/mL at about 2 weeks, about 4 weeks, about 8 weeks, about 12 weeks, about 24 weeks, or about 48 weeks post-treatment. In some aspects the subject has HCV RNA levels below about 25 IU/mL for about 6 months, about 1 year, about 2 years, about 5 years, about 10 years, or longer (either post-transplant or post-treatment).

The methods described herein may further comprise optionally administering to the subject at least one additional anti-HCV agent. Examples of additional anti-HCV agents are provided above.

For example, in various methods, Compound A may also be administered. Compound A can be administered in an amount ranging from about 10 mg/day to about 200 mg/day. For example, the amount of Compound A can be about 30 mg/day, about 45 mg/day, about 60 mg/day, about 90 mg/day, about 120 mg/day, about 135 mg/day, about 150 mg/day, about 180 mg/day. In some methods, Compound A is administered at about 90 mg/day.

The therapeutic agents and combinations thereof disclosed herein are contemplated to exhibit therapeutic activity when administered in an amount that can depend on the particular case. The variation in amount can depend, for example, on the subject being treated and the active ingredients chosen. A broad range of doses can be applicable. Dosage regimens may be adjusted to provide the optimum therapeutic response. For example, several divided doses may be administered daily, weekly, monthly or other at suitable time intervals or the dose may be proportionally reduced as indicated by the exigencies of the situation. Such dosages are optionally altered depending on a number of variables, not limited to the activity of the one or more active ingredients used, the disease or condition to be treated, the mode of administration, the requirements of the individual subject, the severity of the disease or condition being treated, and the judgment of the practitioner.

As described herein, Compound 1 may be administered (or any of the metabolites thereof described herein may be provided) optionally with one or more additional anti-HCV agents. In the various methods described herein, concomitant administration of these agents is contemplated. Concomitant administration refers to the administration of two or more agents (for example, Compound 1 and ribavirin) in any manner in which the pharmacological effects of both agents are manifested in the subject at the same time. Thus, such administration does not require that a single pharmaceutical composition, the same type of formulation, the same dosage form, or even the same route of administration be used for administration of the agents, or that the agents be administered at the same time. Administration of Compound 1 and ribavirin may be concurrent, alternate or any variation thereof, meaning that when the effective amounts of Compound 1 and ribavirin are administered during the same duration, the specific order of administration on a daily basis can be: Compound 1 followed by ribavirin, Compound 1 and ribavirin together, ribavirin followed by Compound 1, or any variation thereof. Additionally, it is contemplated that Compound 1 and ribavirin dosage frequencies may differ. As one non-limiting example, Compound 1 may be administered q.d. while ribavirin is administered b.i.d.

The agents described herein can be formulated into pharmaceutical compositions by combination with appropriate, pharmaceutically acceptable carriers or diluents, and can be formulated into preparations in solid, semi-solid, liquid or gaseous forms, such as tablets, capsules, pills, powders, granules, dragees, gels, slurries, ointments, solutions, suppositories, injections, inhalants and aerosols. As such, administration of the compounds can be achieved in various ways, including oral, buccal, rectal, parenteral, intraperitoneal, intradermal, transdermal, and/or intratracheal administration. Moreover, the compounds can be administered in a local rather than systemic manner, in a depot or sustained release formulation. In addition, the compounds can be administered in a liposome.

The 5′-mono-, di- and/or triphosphate metabolites of Compound 1 may be provided to a subject by administering Compound 1 to the subject, as well as by other means not involving the administration of Compound 1. For example, it is contemplated that the 5′-mono-, di- and/or triphosphate metabolites of Compound 1 may be provided to a subject by administering compounds other than Compound 1 that are converted to the disclosed 5′-mono-, di- and/or triphosphate metabolites in vivo.

Cyclosporine, sirolimus and tacrolimus are immunosuppressants which may be used to prevent allograft rejection in certain subjects. Certain methods described herein do not significantly increase plasma concentrations of an immunosuppressant (such as cyclosporine, sirolimus or tacrolimus), which may also be or have been administered to the subject. For example, certain methods provide for preventing or treating post-liver transplant recurrence of HCV infection wherein the plasma concentrations of an immunosuppressant (such as cyclosporine, sirolimus or tacrolimus) is not significantly increased. Other methods provide for preventing or treating post-liver transplant recurrence of HCV infection wherein the plasma concentrations of an immunosuppressant (such as cyclosporine, sirolimus or tacrolimus) is not increased to levels at which the amount of the immunosuppressant (such as cyclosporine, sirolimus or tacrolimus) would be recommended or instructed to be adjusted.

In another aspect, a package or kit is provided comprising Compound 1 and a package insert, a package label, instructions or other labeling relating to the methods of using Compound 1. Certain packages and kits described herein may include statements relating to no significant increases in plasma levels of cyclosporine, sirolimus or tacrolimus when administered concomitantly with Compound 1. Certain packages and kits described herein require no dose adjustment of an immunosuppressant (such as cyclosporine, sirolimus or tacrolimus). Certain methods, packages and kits described herein require no warning of a drug-drug interaction between Compound 1 and either cyclosporine, sirolimus or tacrolimus. For example, a package or kit can comprise Compound 1 and a package insert or label describing the administration of Compound 1 for preventing or treating post-liver transplant recurrence of HCV infection and statements indicating no significant increases in plasma levels of cyclosporine, sirolimus or tacrolimus when administered with Compound 1.

### EXAMPLES

### Example 1

### Compound 1 and Ribavirin Administered Pre-Transplant to Subjects with HCV to Prevent HCV Recurrence Post-Transplant: Interim Results

### Summary of Study Design, Methods, and Enrollment Criteria

In this open-label study, HCV-infected subjects with hepatocellular carcinoma (HCC) meeting the Milan criteria prior to undergoing liver transplantation were administered a combination of Compound 1 and ribavirin for up to 48 weeks.

During the pre-transplant treatment phase, subjects received a total daily oral dose of 400 mg of Compound 1 q.d. and a total daily oral dose of 1000 mg (weight <75 kg) or 1200 mg (weight ≧75 kg) of ribavirin, divided b.i.d., for a maximum of 48 weeks or until the time of transplant, whichever came first. Treatment was discontinued within 24 hours prior to the liver transplant if transplantation occurred before the subject completed the 48-week treatment course.

After receiving a liver transplant, the subjects entered a post-transplant follow-up phase, wherein they were monitored for evidence of recurrent HCV infection as determined by HCV RNA>LLOQ (i.e., greater than about 25 IU/mL).

Subjects who experienced post-treatment virologic relapse during the pre-transplant treatment phase could, at the clinical investigator's discretion, start a new course of Compound 1+RBV therapy for up to a maximum of 48 weeks of treatment or until transplantation, whichever occurred first.

All subjects met the following criteria, among others: (1) confirmation of chronic HCV infection documented by at least one of (a) a positive anti-HCV antibody test, HCV RNA or HCV genotyping test at least 6 months prior to baseline/day 1 together with a positive HCV RNA test and anti-HCV antibody at the time of screening or (b) a positive HCV RNA test and anti-HCV antibody test at the time of screening together with a liver biopsy consistent with chronic HCV infection; (2) HCV RNA≧10⁴ IU/mL at screening; (3) met the Milan criteria for undergoing liver transplant for HCC secondary to HCV with a MELD score of <22 and an HCC-weighted MELD score of ≧22; and (4) Child-Pugh Score ≦7. All subjects were naïve to treatment with a direct-acting antiviral targeting the HCV NS5b polymerase, received liver transplants only from deceased donors, and had no history of previous solid organ transplantation.

Subjects' HCV RNA levels were measured at the initial screening visit, baseline (day 1 of treatment), weeks 1, 2, 3, 4 and then every 4 weeks through the end of treatment (48 weeks total), at 4 week intervals off-treatment if not yet transplanted, at the time of transplant, and during post-transplant follow-up.

Any subject experiencing virologic failure while receiving active therapy was discontinued from the study. In addition, subjects meeting any of the following criteria were discontinued from therapy: (1) elevated ALT and/or AST>5×baseline or 5×nadir (unrelated to any interventional procedure); (2) elevated ALT>15×ULN (unrelated to any interventional procedure); (3) total bilirubin >6.0 (unrelated to any interventional procedure); (4) any grade 2 or greater rash associated with constitutional symptoms; (5) any non-laboratory grade 4 event assessed as related to treatment with Compound 1/RBV; or (6) worsening disease state as evidence by progressing hepatic decompensation and/or progression of Child-Pugh Score (CPT)≧10.

A subject's drug regimen was modified for any of the following criteria: (1) subject developed a contraindication for transplant during the course of treatment (subject could continue with treatment at the discretion of the clinical investigator); (2) subject's creatinine clearance decreased to below 30 mL/min during drug administration; or (3) subject with persistent hemoglobin of <8.5 g/dL, despite ribavirin dose reduction (in which case the subject could continue with Compound 1 without RBV at the discretion of the clinical investigator).

Virologic failure was defined by: (1) breakthrough (HCV RNA≧25 IU/mL after having previous had HCV RNA<25 IU/mL, while on treatment, confirmed within 1 week); (2) rebound (breakthrough or >1 log₁₀ IU/mL increase in HCV RNA from nadir while on treatment, confirmed within 1 week); (3) non-response (HCV RNA≧25 IU/mL through 8 weeks of treatment); (4) pre-transplant relapse (HCV RNA≧25 IU/mL during the post-treatment period having achieved HCV RNA<25 IU/mL at the end of treatment, confirmed within 1 week); or (5) post-transplant HCV re-occurrence (HCV RNA≧25 IU/mL at any time post liver transplant and confirmed within 1 week).

A subject's ribavirin drug regimen was modified for the following criteria: (1) hemoglobin <10 g/dL for subjects with no cardiac disease (RBV reduced to 600 mg/day); (2) ≧2 g/dL decrease in hemoglobin during any 4-week period of treatment for subjects with a history of stable cardiac disease (RBV reduced to 600 mg/day); (3) hemoglobin <8.5 g/dL for subjects with no cardiac disease (RBV discontinued); (4) hemoglobin <12 g/dL despite 4 weeks at reduced load for subjects with history of stable cardiac disease (RBV discontinued).

HCV RNA was measured using the COBAS® TaqMan® HCV Test, v2.0 for use with the High Pure System.

HCV genotype and subtype were determined using the Siemens VERSANT® HCV Genotype LiPA 2.0 Assay.

IL28B genotype was determined by polymerase chain reaction (PCR) amplification of the SNP, rs12979860, with sequence specific forward and reverse primers and allele specific fluorescently labeled TaqMan® MGB probes.

Creatinine clearance was calculated by the Cockcroft-Gault equation using actual body weight (BW) (Cockcroft D W, Gault M H. Prediction of creatinine clearance from serum creatinine. Nephron 1976; 16:31-41):

$$Male \text{:} {CL}_{cr} ( mL \text{/} min ) = \frac{[ 140 - age ( years ) ] \times BW ( kg )}{72 \times {S}_{cr}}$$

$$Female \text{:} {CL}_{cr} ( mL \text{/} min ) = \frac{[ 140 - age ( years ) ] \times BW ( kg )}{72 \times {S}_{cr}}$$

$${S}_{cr} = serum creatinine ( mg \text{/} dL )$$

### Results

An interim synoptic study report of Study No. P7977-2025 was generated, the results of which are summarized here and in Tables 1-2.

After starting treatment with SOF+RBV, potent and rapid suppression of HCV RNA was observed in the all of the analyzed subjects, with a 3.87 log₁₀ IU/mL mean decrease in HCV RNA after 1 week of treatment and HCV RNA<LLOQ (RNA<25 IU/mL) in 93.1% of subjects by Week 4 of SOF+RBV treatment. With the exception of 5 subjects who had on-treatment virologic failures, all subjects had HCV RNA<LLOQ for the duration of treatment or until the time of liver transplantation.

A total of 8 of 12 subjects who completed 24 weeks of treatment and had a Week 4 post-treatment follow-up visit relapsed in the pre-transplant phase and were eligible to restart treatment in the retreatment study.

Approximately 90% of the subjects transplanted went into the liver transplantation with HCV RNA<LLOQ as presented in the Table 1:

TABLE 1

| | SOF + RBV |
|---|---|
| Number of Subjects Transplanted | 28 |
| Number of Subjects with ≧12 Weeks of Treatment and | 17 |
| Received a Liver Transplantation^{a} | |

<LLOQ at Last HCV RNA Measurement Prior to Liver Transplantation

| | |
|---|---|
| Yes | 15/17 (88.2%) |
| No | 2/17 (11.8%) |
| Number of Subjects with Any Treatment Duration and | 28 |
| Received a Liver Transplantation^{b} | |

<LLOQ at Last HCV RNA Measurement Prior to Liver Transplantation

| | |
|---|---|
| Yes | 26/28 (92.9%) |
| No | 2/28 (7.1%) |

^{a}The denominator includes all subjects who received a liver transplantation and had ≧12 weeks of study treatment for the primary efficacy analysis set.
^{b}The denominator includes all subjects who received a liver transplantation and had ≧1 dose of study treatment for the secondary efficacy analysis set.

As of this interim report, there appeared to be very little difference in the rate of recurrence for subjects who were <LLOQ at liver transplantation and received at least 12 weeks of SOF+RBV treatment, and those who were <LLOQ and received any duration of treatment with SOF+RBV. Posttransplant virologic response by visit for the FAS with ≧12 weeks of treatment and HCV RNA<LLOQ at the last measurement prior to liver transplantation is presented in the Table 2

TABLE 2

SOF + RBV
(N = 15)

Posttransplant Week 1

| | |
|---|---|
| <LLOQ | 13/15 (86.7%) |
| 90% CI | 63.7-97.6% |

Posttransplant Week 2

| | |
|---|---|
| <LLOQ | 12/15 (80.0%) |
| 90% CI | 56.0-94.3% |

Posttransplant Week 4

| | |
|---|---|
| <LLOQ | 9/12 (75.0%) |
| 90% CI | 47.3-92.8% |

Posttransplant Week 8

| | |
|---|---|
| <LLOQ | 3/5 (60.0%) |
| 90% CI | 18.9-92.4% |

Posttransplant Week 12

| | |
|---|---|
| <LLOQ | 2/4 (50.0%) |
| 90% CI | 9.8-90.2% |

a. HCV RNA analyzed using a Roche Cobas ® Taqman ® Version 2.0 assay for use with High Pure system with limit of quantitation of 25 IU/mL .
b. A missing value at a visit was imputed as ‘<LLOQ’ if it was bracketed by values of ‘<LLOQ detected’ or ‘<LLOQ TND’, otherwise, imputed as ≧LLOQ. Subjects discontinued from study with HCV recurrence imputed to ≧LLOQ at a visit if (extract date - transplantation date + 1) greater than the upper bound of the visit window.
c. Subjects with last HCV RNA prior to transplantation ≧LLOQ or transplanted with an HCV-infected liver were excluded from the posttransplant endpoints.
d. The 90% CI for the proportion of subjects with HCV RNA <LLOQ was based on the Clopper-Pearson method.

### Example 2

### Compound 1 and Ribavirin Administered Pre-Transplant to Subjects with HCV to Prevent HCV Recurrence Post-Transplant

### Methods

Patients were enrolled at 14 centers in the United States, New Zealand (1), and Spain (1). Eligible patients were at least 18 years old with a body mass index of ≧18 kg/m² and documented HCV infection of any genotype with an HCV RNA value >10⁴ IU/mL. Patients who had failed previous treatment for HCV were eligible. Patients were required to be on the waiting list for liver transplantation (with anticipated time until transplantation of less than 1 year) from a deceased donor. Patients had hepatocellular carcinoma (HCC) meeting the Milan criteria²¹ secondary to HCV, with a biological Model for End-Stage Liver Disease (MELD) score of <22, and a Child-Turcotte-Pugh score of ≦7 and had to be eligible for a MELD exception score as per policy of the United Network for Organ Sharing. We chose to study patients with HCC because they could be expected to undergo liver transplantation within 1 year.

This phase 2, open-label pilot study had 2 phases: a pre-transplant treatment phase and a post-transplant follow-up phase. During the pre-transplant treatment phase, patients received sofosbuvir (Compound 1) administered orally at a dose of 400 mg once daily, along with ribavirin (Ribasphere, Kadmon) administered orally as a divided dose according to body weight (1000 mg daily in patients with a body weight of <75 kg, and 1200 mg daily in patients with a body weight of ≧75 kg). According to the original study protocol, treatment lasted up to 24 weeks or until time of transplant, whichever occurred first. Patients who completed treatment prior to transplantation were also assessed for sustained virologic response. Patients who relapsed after stopping study drug during the pre-transplant treatment phase and who were not found to have the S282T NS5B mutation (which is associated with resistance to sofosbuvir) were allowed to restart treatment and continue for an additional 24 weeks or until transplant. In a subsequent amendment, the study design was changed to allow all patients who had not reached 24 weeks of treatment at the time of the amendment to continue treatment, uninterrupted to 48 weeks or transplant. This change was made after observing virologic relapse in 3 patients prior to transplantation of those who stopped treatment after completing 24 weeks.

For those still receiving treatment at the time of transplantation, dosing was discontinued within 24 hours prior to transplant. During the post-transplant follow-up phase, patients were followed for 48 weeks for evidence of recurrent HCV infection. All participating sites planned to use a standard post-transplantation immunosuppressive regimen of solumedrol/prednisone, tacrolimus and/or mycophenolate mofetil (up to 2 g a day) for the first 12 weeks after transplantation. Antibody induction (monoclonal antibodies; e.g. infliximab) was prohibited during the study.

The primary efficacy end point was post-transplantation virologic response (pTVR), defined as HCV RNA less than the lower limit of quantification (LLOQ, 25 IU/mL) at 12 weeks post-transplant in patients who had HCV RNA<LLOQ at their last assessment prior to transplantation. According to the original study analysis plan, only patients who received at least 12 weeks of treatment before transplantation were to be included in the efficacy analysis. However, this restriction was not used in the analysis so that the efficacy population includes patients who received any duration of treatment (overall results for both populations are included in Table 3). Other secondary efficacy end points included an evaluation of safety and tolerability. Plasma HCV RNA levels were measured with the COBAS TaqMan HCV Test, version 2.0, for use with the High Pure System (Roche Molecular Systems).

### Results

In the 61 subjects who received study drug the median duration of exposure to study drugs was 21 weeks, with a range of 2.3 to 52.3 weeks. Treatment with sofosbuvir and ribavirin resulted in rapid suppression of circulating virus with a median decrease in HCV RNA of 3.93 log 10 IU/mL after 1 week of treatment. By the fourth week of treatment, 54 of the 58 patients (93%) receiving treatment had HCV RNA<LLOQ. The rate and amount of decline in HCV RNA levels did not differ by prior HCV treatment history or CTP class.

Of the 46 patients who underwent transplantation, 43 had HCV RNA<LLOQ at the time of transplantation and represent the pre-specified group for which we determined efficacy of treatment. Of these, 29/42 (69%) have achieved pTVR12 (Table 3). One patient has not reached the post-transplantation week 12 timepoint. Of the 13 not achieving pTVR12, 10 had confirmed HCV recurrence and 3 died immediately post-transplant. Expressed as a percentage of the total population who received study treatment, 48% (29 of 61) achieved pTVR.

TABLE 3
Post-transplant virologic response by visit for patients with HCV RNA
<LLOQ at the last measurement prior to liver transplantation

| | |
|---|---|
| Sofosbuvir-ribavirin for | Sofosbuvir-ribavirin for |
| ≧12 weeks (N = 32) | any duration (N = 43) |

Post-transplant Week 1

| | | |
|---|---|---|
| <LLOQ, n/N (%) | 28/32 (88%) | 37/43 (86%) |
| 90% CI | 74-96% | 74-94% |

Post-transplant Week 2

| | | |
|---|---|---|
| <LLOQ, n/N (%) | 25/31 (81%) | 34/42 (81%) |
| 90% CI | 65-91% | 68-90% |

Post-transplant Week 4

| | | |
|---|---|---|
| <LLOQ, n/N (%) | 23/31 (74%) | 30/42 (71%) |
| 90% CI | 58-86% | 58-83% |

Post-transplant Week 8

| | | |
|---|---|---|
| <LLOQ, n/N (%) | 23/31 (74%) | 30/42 (71%) |
| 90% CI | 58-86% | 58-83% |

Post-transplant Week 12

| | | |
|---|---|---|
| <LLOQ, n/N (%) | 23/31 (74%) | 29/42 (69%) |
| 90% CI | 58-86% | 55-81% |

Note:
HCV RNA analyzed using Roche Taqman Version 2.0 assay for use with High Pure system with limit of quantification of 25 IU/mL.

### Discussion

In this pilot study, sofosbuvir and ribavirin before liver transplantation prevented recurrence of HCV infection in approximately two thirds of patients with chronic HCV infection and liver cancer who achieved an HCV RNA<25 IU/mL prior to transplantation and in almost half of the total patients in the study. This population of patients with compensated or mildly decompensated cirrhosis included those with characteristics historically associated with lower rates of response to antiviral therapy—high viral load, non-CC genotype and prior non-response to interferon therapy. The rate of discontinuation due to adverse events was low, and most observed events were those commonly associated with ribavirin therapy—fatigue, anemia, headache, and nausea—as were the laboratory abnormalities of decreased hemoglobin and elevated bilirubin.

This study provides proof of concept that virologic suppression without interferon can significantly reduce the rate of recurrent HCV after liver transplantation.

### Example 3

### Compound 1 and Ribavirin for 24 Weeks in Subjects with Recurrent Chronic HCV Post-Liver Transplant: Interim Results

### Study Design, Methods, and Enrollment Criteria

In this open-label study, subjects were administered a total daily oral dose of 400 mg of Compound 1 q.d. and an initial ribavirin total daily oral dose starting at 400 mg, divided b.i.d. The ribavirin dose could be increased at weeks 2, 4 and monthly by 200 mg/day/month, to a maximum ribavirin dose of 1000 mg/day or 1200 mg/day depending on the subject's body weight. A subject's ribavirin dosage was increased based on the subjects' hemoglobin value (Hb) and creatinine clearance. If the subject's Hb≧12 g/dl, the subject's ribavirin dose was increased by 200 mg/day/at weeks 2, 4 and monthly until a full dose of 1000 mg (weight <75 kg) or 1200 mg (weight ?: 75 kg) per day was reached. If a subject's Hb was 10-12 g/dl, the subject's current ribavirin dose was maintained. If a subject's Hb was 8-10 g/dl, the subject's ribavirin dose was reduced by 200 mg/day (with growth factors allowed, if medically necessary). The total duration for treatment with a combination of Compound 1+RBV was 24 weeks.

All subjects had received a liver transplant ≧6 months and ≦150 months prior to screening; demonstrated an absence of organ rejection as documented by a post-transplant liver biopsy taken within 12 months prior to baseline/day 1 of treatment; received a primary or secondary (re-transplant) liver alone or liver and kidney transplant (and were not recipients of a multi-organ transplant including heart or lung); had evidence of chronic HCV infection pre-transplantation documented by either (a) a positive anti-HCV antibody test, HCV RNA or HCV genotyping test, or (b) a liver biopsy performed with evidence of chronic HCV disease, such as the presence of fibrosis and no evidence of rejection; and had HCV RNA≧10⁴ IU/mL at screening. All subjects were naïve to nucleotide/nucleoside treatment for chronic HCV infection and had not been exposed to protease inhibitors within three months prior to the baseline/day 1 visit.

Subjects' HCV RNA levels were measured at the initial screening visit, baseline (day 1 of treatment), day 3 of treatment, weeks 1, 2, 3, 4 and then every 4 weeks through the end of treatment (24 weeks total), and then at 2, 4, 8, 12, 24 and 48 weeks following the last dose of study drug. For this study, SVR-12 was defined as HCV RNA<LLOQ (i.e., <about 25 IU/mL) twelve weeks after the last dose of the study drug.

The following on-treatment virologic response-based treatment stopping criteria were used for all subjects: (1) confirmed HCV RNA≧LLOQ after 2 consecutive HCV RNA<LLOQ; (2) confirmed >1 log 10 increase from nadir; or (3) HCV RNA≧LLOQ through 8 weeks of treatment. Subjects meeting any of the following criteria were discontinued from therapy: (1) confirmed elevation of serum alanine aminotransferase (ALT) and/or aspartate aminotransferase (AST)>5×baseline or 5×nadir (unrelated to any interventional procedure); (2) confirmed elevation of ALT>15×upper limit of normal range (ULN) (unrelated to any interventional procedure); (3) confirmed total bilirubin >10.0×ULN (unrelated to any interventional procedure); (4) confirmed total bilirubin >3× baseline or 3× nadir (unrelated to any interventional procedure); (5) any Grade 2 or greater rash associated with constitutional symptoms; (6) any non-laboratory Grade 4 event assessed as related to treatment with Compound 1/RBV; (7) worsening of disease state as evidenced by progressing hepatic decompensation and/or progression of Child-Pugh (CPT) Score to ≧10; or (8) steroid-resistant acute allograft rejection.

A subject's ribavirin drug regimen was modified for either of the following criteria: (1) creatinine clearance that decreased to below 30 mL/min during drug administration, or (2) persistent hemoglobin of <8.5 g/dL, despite ribavirin dose reduction (in which case the subject could continue with Compound 1 without RBV at the discretion of the clinical investigator). Ribavirin dosing was stopped for any of the following: (1) Hb<8 g/dl despite growth factor use; (2) rising bilirubin level with direct fraction >10 mg/dl; or (3) creatinine >2.5 mg/dl.

HCV RNA was measured using the COBAS® TaqMan® HCV Test, v2.0 for use with the High Pure System.

HCV genotype and subtype were determined using the Siemens VERSANT® HCV Genotype INNO-LiPA 2.0 Assay.

IL28B genotype was determined by polymerase chain reaction (PCR) amplification of the SNP, rs12979860, with sequence specific forward and reverse primers and allele specific fluorescently labeled TaqMan® MGB probes.

Creatinine clearance was calculated by the Cockcroft-Gault equation using actual body weight (BW) (Cockcroft D W, Gault M H. Prediction of creatinine clearance from serum creatinine. Nephron 1976; 16:31-41):

$$Male \text{:} {CL}_{cr} ( mL \text{/} min ) = \frac{[ 140 - age ( years ) ] \times BW ( kg )}{72 \times {S}_{cr}}$$

$$Female \text{:} {CL}_{cr} ( mL \text{/} min ) = \frac{[ 140 - age ( years ) ] \times BW ( kg ) \times 0.85}{72 \times {S}_{cr}}$$

$${S}_{cr} = serum creatinine ( mg \text{/} dL )$$

### Results

To date, 37 of 40 total subjects have been enrolled and have received a mean duration of treatment of 9.8 weeks (2.4-20.4 weeks). Thirty-six subjects remain on treatment and 1 subject has discontinued due to an adverse event (AE) of pneumonia, which was not related to study treatment. Subjects' mean baseline HCV RNA was 6.51 log₁₀ IU/mL (4.49-7.59 log₁₀ IU/mL) By Weeks 3 and 4 on treatment, 86% and 100% of evaluable subjects, respectively, had HCV RNA<25 IU/mL. Treatment with sofosbuvir and ribavirin has been well tolerated. Two subjects have experienced treatment-emergent SAEs not related to sofosbuvir, pyrexia and pneumonia. Twenty-four subjects (65%) experienced treatment-emergent, treatment-related AEs. The most common treatment-related AEs were headache, nausea, fatigue, dyspnea, and anemia.

### Example 4

### Compound 1 and Ribavirin for 24 Weeks in Subjects with Recurrent Chronic HCV Post-Liver Transplant

This was a multi-center, open-label study. Patients received sofosbuvir 400 mg and ribavirin 200-1200 mg orally every day for 24 weeks. After treatment, patients were followed for up to 48 weeks. The initial dose of ribavirin was 400 mg/day in two divided doses. If hemoglobin was >12 g/dL, ribavirin was increased as tolerated by 200 mg/day at Weeks 2, 4, and up to every 4 weeks until the full dose (1000-1200 mg/day) was reached. If hemoglobin was 10-12 g/dL, existing dosing was maintained. And if hemoglobin was 8-10 g/dL, ribavirin dose was reduced by 200 mg/day. Ribavirin dosing was stopped if hemoglobin was <8 g/dL despite growth factor use, if bilirubin level was rising and had a direct fraction >10 mg/dL, or creatinine >2.5 mg/dL.

Treatment was to be stopped for patients with the following virologic criteria: confirmed HCV RNA≧the lower limit of quantification (LLOQ) after 2 consecutive HCV RNA<LLOQ, confirmed HCV RNA>1 log₁₀ increase from nadir, or HCV RNA≧LLOQ through 8 weeks of treatment. In addition, treatment was to be stopped for patients with any of the following safety reasons: ALT or AST>5×baseline; ALT>15×ULN; total bilirubin >10.0×ULN; total bilirubin >3×baseline or 3×nadir; any Grade 2 or greater rash associated with constitutional symptoms; any non-laboratory Grade 4 event assessed as related to study treatment; progressing hepatic decompensation; or steroid-resistant acute allograft rejection.

Eligible patients had chronic HCV (all genotypes) and were at least 18 years old, had HCV RNA≧10⁴ IU/mL, and had received a primary or secondary liver or liver and kidney transplant from a deceased or living donor. Liver transplants took place ≧6 months and <150 months prior to screening. Absence of organ rejection was documented by a post-transplant liver biopsy taken within 12 months of the first on-study treatment dose. Patients had Child-Pugh-Turcotte Scores <7 and MELD scores <17. Patients with any of the following conditions or characteristics were excluded from participation: decompensated liver disease; heart or lung transplant; use of corticosteroids at any dose equivalent to >5 mg of prednisone/day; HIV infection; serum creatinine >2.5×ULN; white blood cells >20×10⁹/L; absolute neutrophil count <1,000 cells/mm³; hemoglobin <10 g/dL; platelets <25,000/mm³; bilirubin ≧4×ULN; alanine aminotransferase (ALT), aspartate aminotransferase (AST), or alkaline phosphatase ≧10×ULN; or use of T-cell depleting or masking antibodies, systemic antineoplastic agents, cyclosporine >300 mg/day, sirolimus, or everolimus. All patients provided written informed consent before undertaking any study-related procedures.

### Methods

Plasma samples for determining HCV RNA levels were drawn at Screening; on days 1 and 3 of treatment; at weeks 1, 2, 3, 4, 8, 12, 16, 20, and 24 of treatment; and at follow-up weeks 2, 4, 8, 12, 24, and 48. Plasma HCV RNA was analyzed by using the Roche COBAS TaqMan HCV Test, v2.0 for Use with the High Pure System (Roche Molecular Systems, Inc., Branchburg, N.J.), which has a lower limit of quantification of 25 IU/mL.

The primary efficacy endpoint was the proportion of subjects with SVR12, defined as HCV RNA<LLOQ (25 IU/mL) 12 weeks after stopping study drug. Along with the proportion of patients with SVR12, a 2-sided exact 90% confidence interval was constructed by using the Clopper-Pearson method. The study was powered such that if an SVR12 rate of 50% was observed, a sample size of 40 would be sufficient to show that the lower bound of a 1-sided exact 95% CI (using the Clopper-Pearson method) is 36.1%.

The secondary efficacy endpoint was the proportion of patients with HCV RNA<LLOQ over time.

The primary safety endpoint was the proportion of patients who discontinued from study drug because of an adverse event.

### Results

At 12 international study sites (8 in the United States, 1 in Germany, 1 in France, 1 in New Zealand, and 1 in Spain), 40 patients initiated study treatment. A majority of patients were white (85%), male (78%), or had undergone prior HCV treatment (88%) (Table 1). The median (range) patient age was 59 (49-75) years. Genotype 1 HCV was present in 33 patients (83%), and HCV RNA levels were ≧6 log₁₀ IU/mL in 32 patients (80%). At baseline, 16 patients (40%) had cirrhosis (METAVIR F4). On average, patients had undergone liver transplantation 4.8 years prior. Greater than 70% of patients had a MELD score ≦10 at screening. The majority of patients were receiving the immunosuppressant tacrolimus (70%) for maintenance immunosuppression.

Thirty-eight patients (95%) completed 24 weeks of study treatment; 2 discontinued study treatment early because of an adverse event but remained on study for follow-up efficacy visits. Twenty-eight patients (70%) completed the study through the primary endpoint without relapse; 11 discontinued the study because of post-treatment relapse, and 1 was lost to follow-up but confirmed by the site to have relapsed.

All patients receiving sofosbuvir and ribavirin were HCV RNA negative by week 4 of therapy. Virologic response continued throughout treatment; all 40 patients had HCV RNA<LLOQ at their last treatment visit, and the 38 patients who completed 24 weeks of therapy had HCV RNA<LLOQ at the end of therapy (Table 4). During the follow-up period, 11 patients experienced observed relapse, and an additional subject was confirmed by the site to have relapsed. Of the 12 subjects who had confirmed relapse; 7 relapsed at follow-up Week 2, 4 at follow-up Week 4 and one at follow-up Week 12. A total of 28 patients (70% of those who initiated therapy; 90% CI, 56% to 82%) reached the primary endpoint of SVR12. These 28 patients also had HCV RNA<LLOQ at 24 weeks after stopping therapy.

TABLE 4
Antiviral Response^{a}

Sofosbuvir + Ribavirin
N = 40

During Treatment, n

| | |
|---|---|
| Week 2 | 23 (58%) |
| Week 4 | 40 (100%) |
| Week 12 | 40 (100%) |
| Week 24 | 38 (95%)^{b} |

After Treatment, n

| | |
|---|---|
| Week 2 | 33 (83%) |
| Week 4 | 29 (73%) |
| Week 8 | 29 (73%) |
| Week 12 | 28 (70%) |
| Week 24 | 28 (70%) |

^{a}Hepatitis C virus RNA < 25 IU/mL
^{b}Two patients had discontinued treatment because of adverse events (pneumonia, hepatocellular carcinoma) and did not have HCV RNA collected at week 24. Both patients who discontinued treatment early had HCV RNA < 25 IU/mL at the time of discontinuation.

### Example 5

### Compound 1, Compound A, and Ribavirin for the Treatment of HCV in Patients with Post Transplant Recurrence

### Example 5

Compound 1, Compound A, and Ribavirin for the Treatment of HCV in Patients with Post Transplant Recurrence

Methods:

GT 1 and 4, naïve and treatment-experienced patients with HCV infection, who were post liver transplantation (fibrosis score 0-3, CPT class A, B and C cirrhosis) with an estimated glomerular filtration rate (GFR) >40 mL/min, received 12 or 24 weeks of Compound 1, Compound A, and RBV. The primary efficacy endpoints were SVR (HCV RNA<25 IU/mL) 12 weeks after completion of study treatment, safety and tolerability.

Results:

To date, 223 patients have been randomized and treated. Most were male (83%), Caucasian (87%), and had prior HCV treatment (83%). The median time since liver transplant was 4.4 years (0.4-233). Mean baseline HCV RNA was 6.4 log₁₀ IU/mL [range 2.4-7.8 log₁₀ IU/mL]. Mean GFR was 65.5 [range 20.4-118.9 mL/min]. 112 patients had F0-F3 fibrosis, 52, 50 and 9 patients had CPT class A, B, and C cirrhosis, respectively. Interim Observed SVR4 results are depicted in Table 1.

TABLE 5
Interim Observed SVR4 Results

| | Post Transplant | | Post Transplant | |
|---|---|---|---|---|
| | F0-F3 | | CPT A, B and C Cirrhosis | |
| | Compound 1, | Compound 1, | Compound 1, | Compound 1, |
| | Compound A, | Compound A, | Compound A, | Compound A, |
| | RBV 12 weeks | RBV 24 weeks | RBV 12 weeks | RBV 24 weeks |
| n/N (%) | (N = 55) | (N = 57) | (N = 57) | (N = 54) |
| SVR4 | 51/53 (96) | 15/16 (94) | 43/47 (91) | 9/11 (82) |

The most common adverse events were fatigue, anemia, headache and nausea. 9 SAEs in 8 patients were considered related to study treatment; anemia (4) and hemolytic anemia (2), sick sinus syndrome (1), sinus arrhythmia (1) and portal vein thrombosis (1). Five patients with cirrhosis died while in the study due to internal bleeding, multiorgan failure/intestinal perforation, cardiac, complications of cirrhosis and progressive multifocal leukoencephalitis. Median serum creatinine and INR remained at baseline levels throughout treatment. Consistent with patients who have moderate renal impairment and who are receiving RBV, hemoglobin values decreased 2-3 g/dL while on treatment. 32 patients received epoetin or blood transfusions while in the study.

While the foregoing description describes specific aspects, embodiments, and examples, those with ordinary skill in the art will appreciate that various modifications and alternatives can be developed. Accordingly, the particular aspects, embodiments, and examples described above are meant to be illustrative only, and not to limit the scope of the invention, which is to be given the full breadth of the appended claims, and any and all equivalents thereof.

We claim:

1. A method of preventing or reducing the risk of post-liver transplant recurrence of a hepatitis C virus infection comprising administering to a subject an effective amount of Compound 1.

2. The method of claim 1, wherein Compound 1 is administered for a pre-transplant duration, wherein the pre-transplant duration is from about 1 week to about 48 weeks.

3. The method of claim 1, wherein Compound 1 is administered for a post-transplant duration, wherein the post-transplant duration is from about 1 week to about 48 weeks.

4. The method of claim 1, further comprising administering to the subject an effective amount of ribavirin, wherein the effective amount of Compound 1 is about 400 mg per day and the effective amount of ribavirin is from about 1000 mg to about 1200 mg per day.

5. A method of preventing or reducing the risk of post-liver transplant recurrence of a hepatitis C virus infection comprising providing to a subject a 5′-mono-, di- or triphosphate metabolite of Compound 1.

6. A method of reducing HCV RNA levels to lower than about 25 IU/mL in a subject having received a liver transplant comprising administering to the subject an effective amount of Compound 1.

7. The method of claim 6, wherein the effective amount of Compound 1 is administered for a pre-transplant duration, wherein the pre-transplant duration is from about 1 week to about 48 weeks.

8. The method of claim 7, wherein the pre-transplant duration is from about 12 weeks to about 48 weeks.

9. The method of claim 6, wherein the effective amount of Compound 1 is administered for a post-transplant duration, wherein the post-transplant duration is from about 1 week to about 48 weeks.

10. The method of claim 9, wherein the pre-transplant duration is from about 12 weeks to about 48 weeks.

11. The method of claim 6, wherein the effective amount of Compound 1 is about 400 mg per day.

12. The method of claim 6, further comprising administering to the subject an effective amount of ribavirin.

13. The method of claim 12, wherein the effective amount of ribavirin is from about 1000 mg to about 1200 mg per day.

14. The method of claim 12, wherein the effective amount of Compound 1 is about 400 mg per day and the effective amount of ribavirin is from about 1000 mg to about 1200 mg per day.

15. A method of reducing HCV RNA levels to lower than about 25 IU/mL in a subject having received a liver transplant comprising providing to the subject a 5′-mono-, di- or triphosphate metabolite of Compound 1.

16. The method of claim 6, further comprising administering at least one additional antiviral agent to the subject.

17. The method of claim 16, wherein the additional antiviral agent is an HCV NS5A protein inhibitor, a non-nucleotide NS5B polymerase inhibitor, or an NS3 inhibitor.

18. The method of claim 17, wherein the at least one additional antiviral agent is Compound A, or Compound B.

19. The method of claim 18, wherein the at least one additional antiviral agent is Compound A.

20. The method of claim 19, further comprising administering ribavirin to the subject.

21. The method claim 6, wherein the subject has less than about 25 IU/mL of HCV RNA at 4 weeks post-treatment.

22. The method of claim 6, wherein the subject has less than about 25 IU/mL of HCV RNA at 12 weeks post-treatment.
